(19) United States
(12) Patent Application Publication
(10) Pub. No.: US 2017/0340217 A1
(43) Pub. Date: Nov. 30, 2017

(54) PHYSIOLOGICAL DETECTION DEVICE

(71) Applicant: Leadtek Research Inc., New Taipei City (TW)

(72) Inventors: Po-Chun Hsu, New Taipei City (TW); Han-Wen Hu, Taipei City (TW); Hsien-Chih Ou, New Taipei City (TW); Chi-Hua Chan, Taipei City (TW); Yun-Yi Huang, Taipei City (TW)

(73) Assignee: Leadtek Research Inc., New Taipei City (TW)

(21) Appl. No.: 15/296,014

(22) Filed: Oct. 17, 2016

(30) Foreign Application Priority Data
May 30, 2016 (TW) 105207982

(51) Int. Cl. A61B 5/0295; A61B 5/026; A61B 5/1455; A61B 5/00

(52) U.S. Cl. CPC A61B 5/0295; A61B 5/7225; A61B 5/0261; A61B 5/14551; A61B 5/6826

(57) ABSTRACT

A physiological detection device includes a main body, a sensor pair, a signal processor, and a calculation module. The sensor pair is disposed in the main body and adapted to detect a detected portion of a human body, so as to obtain a sensing signal. The signal processor is disposed in the main body and receives and processes the sensing signal, so as to output a digital physiological signal. The calculation module receives the digital physiological signal and calculates to obtain first information and second information of a plurality of feature points of the digital physiological signal. The calculation module calculates a ratio of the second information to the first information, so as to obtain a physiological state index. The digital physiological signal includes a plurality of pulse waves generated according to a time sequence.

### CROSS-REFERENCE TO RELATED APPLICATION

This application claims the priority benefit of Taiwan application serial no. 105207982, filed on May 30, 2016. The entirety of the above-mentioned patent application is hereby incorporated by reference herein and made a part of this specification.

### BACKGROUND OF THE INVENTION

### Field of the Invention

The invention relates to a physiological detection device and particularly relates to a physiological detection device for detecting a body circulation state.

### Description of Related Art

Cardiovascular disease has become one of the major causes of death around the world. Therefore, the research and development of various detection methods for cardiovascular circulation have become more important than before. Among current detection methods, the method of detecting peripheral blood circulation based on photoplethysmography signals generated by a photoplethysmograph (PPG) is drawing more and more attention. The PPG is capable of obtaining an optical volume pulse of the blood in the detected portion of the human body so as to calculate a physiological state index by using a calculator according to the obtained optical volume pulse wave.

Specifically, a physiological detection device that uses the PPG to detect and measure the circulation state calculates the physiological state index based on information of feature points of the optical volume pulse wave signal obtained from the detected portion of the human body. FIG. 1 is a pulse waveform diagram showing a volume pulse wave of a digital physiological signal from the conventional physiological detection device. Referring to FIG. 1, a conventional method of calculating the physiological state index is to calculate a reflection index according to a ratio of a height difference a between a trough point d3 and a pulse peak d1 (i.e., a systolic wave pulse peak) of the pulse wave to a height difference b between the trough point d3 and a diastolic wave peak d2. In the conventional calculation method, a ratio of the height of the subject to a time difference Td between the systolic pulse peak d1 and the diastolic wave peak d2 may also be calculated to serve as a stiffness index.

However, the way the conventional physiological detection device calculates the physiological state index has the following deficiency. Specifically, an optical volume pulse signal of a normal subject has a pulse wave with a transient rebound and rise during the process of descending, which is the above-mentioned diastolic wave. For subjects who are in poor health or aging, however, the optical volume pulse wave signal obtained by detecting the detected portion may not have the diastolic wave or the diastolic wave may not have an obvious diastolic peak. Consequently, the conventional physiological detection device may not be able to effectively obtain the physiological state index of the subject by the aforementioned calculation method. For the above reason, the physiological state index detection and calculation method of the conventional physiological detection device are not applicable to all subjects. Thus, it has become an important issue in this field to develop a physiological detection device that can easily and accurately detect body circulation for all subjects.

### SUMMARY OF THE INVENTION

The invention provides a physiological detection device that detects and assesses a body circulation state of a subject in a noninvasive manner.

The physiological detection device of the invention includes a main body, a sensor pair, a signal processor, and a calculation module. The sensor pair is disposed in the main body and adapted to detect a detected portion of a human body to obtain a sensing signal. The signal processor is disposed in the main body and receives and processes the sensing signal to output a digital physiological signal. The calculation module receives the digital physiological signal and calculates to obtain first information and second information of a plurality of feature points of the digital physiological signal. The calculation module calculates a ratio of the second information to the first information to obtain a physiological state index. The digital physiological signal includes a plurality of pulse waves generated according to a time sequence. The feature points of the digital physiological signal include a pulse peak of each of the pulse waves and a foot point at a forepart of a rising edge of each of the pulse waves.

In an embodiment of the invention, the first information is an integral area of the pulse wave between the foot point and the pulse peak with respect to a time axis while the second information is an integral area of the pulse wave between adjacent two foot points with respect to the time axis.

In an embodiment of the invention, the first information is a time difference between the foot point and the pulse peak while the second information is a time difference between the adjacent two foot points.

In an embodiment of the invention, the sensor pair is a photoplethysmograph, including an optical emitter and an optical receiver. The optical emitter emits a light that passes through the detected portion of the human body. The optical receiver receives the light passing through the detected portion to obtain the sensing signal.

In an embodiment of the invention, the signal processor includes a filter, an amplifier, and an analog-to-digital converter. The filter performs filtering on the sensing signal. The amplifier amplifies the sensing signal. The analog-to-digital converter converts the sensing signal into the digital physiological signal.

In an embodiment of the invention, the calculation module includes a normalization processor and a physiological state index calculator. The normalization processor normalizes the digital physiological signal. The physiological state index calculator calculates the physiological state index according to the feature points of the normalized digital physiological signal.

In an embodiment of the invention, the physiological detection device further includes an alarm disposed in the main body and electrically connected with the calculation module.

In an embodiment of the invention, the physiological detection device further includes a display disposed on a surface of the main body and displaying the physiological state index.

In an embodiment of the invention, the physiological detection device further includes a power supply disposed in the main body. The power supply is electrically connected with the sensor pair, the signal processor, and the calculation module.

In an embodiment of the invention, the physiological detection device further includes a transmitter disposed in the main body and transmitting the physiological state index outside the physiological detection device.

Based on the above, the physiological detection device in the embodiments of the invention is capable of detecting the circulation state of the detected portion of the human body. Specifically, the sensor pair of the physiological detection device detects the detected portion of the human body to obtain the sensing signal of the detected portion. The sensing signal is further processed by the signal processor for the digital physiological signal to be outputted. Moreover, the calculation module calculates to obtain multiple feature points from the digital physiological signal and obtains the physiological state index according to the information of the feature points of the digital physiological signal. In the embodiments of the invention, the physiological state of the human body is assessed simply based on the physiological state index obtained by the physiological detection device. Thus, the time, procedure, equipment, and costs required for the general physiological detection are reduced.

To make the aforementioned and other features and advantages of the invention more comprehensible, several embodiments accompanied with drawings are described in detail as follows.

### BRIEF DESCRIPTION OF THE DRAWINGS

The accompanying drawings are included to provide a further understanding of the invention, and are incorporated in and constitute a part of this specification. The drawings illustrate exemplary embodiments of the invention and, together with the description, serve to explain the principles of the invention.

FIG. 1 is a pulse waveform diagram showing a volume pulse wave of a conventional digital physiological signal.

FIG. 2 is a block diagram of a physiological detection device according to an embodiment of the invention.

FIG. 3A is a schematic view of the physiological detection device of FIG. 2.

FIG. 3B is a schematic side view of the physiological detection device of FIG. 3A.

FIG. 3C is a schematic side view of the physiological detection device of FIG. 3A from another aspect.

FIG. 4A to FIG. 4C are pulse waveform diagrams showing a volume pulse wave of a digital physiological signal of the physiological detection device of FIG. 2.

FIG. 5A to FIG. 5F are schematic views of appearance of the physiological detection device according to another embodiment of the invention.

### DESCRIPTION OF THE EMBODIMENTS

Some other embodiments of the invention are provided as follows. It should be noted that the reference numerals and part of the contents of the previous embodiment are used in the following embodiments, in which identical reference numerals indicate identical or similar components, and repeated description of the same technical contents is omitted. Please refer to the description of the previous embodiment for the omitted contents, which will not be repeated hereinafter.

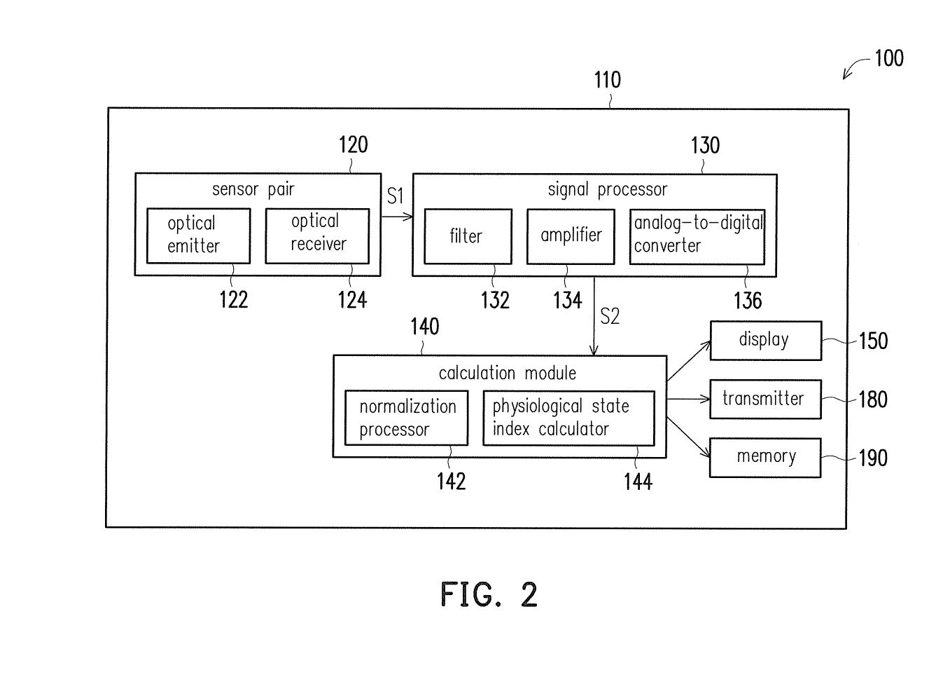

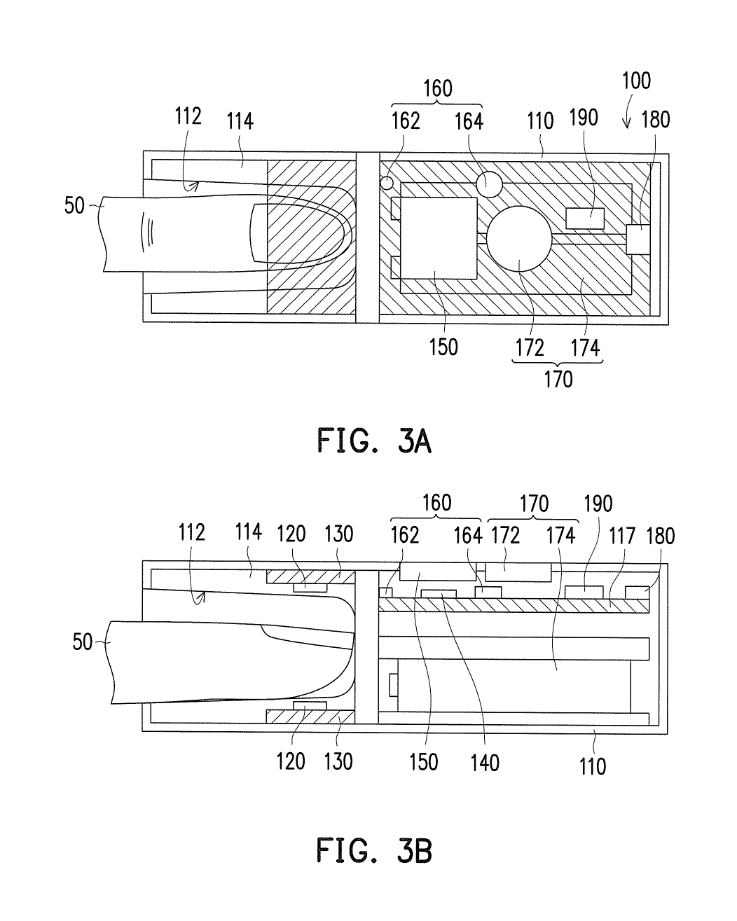

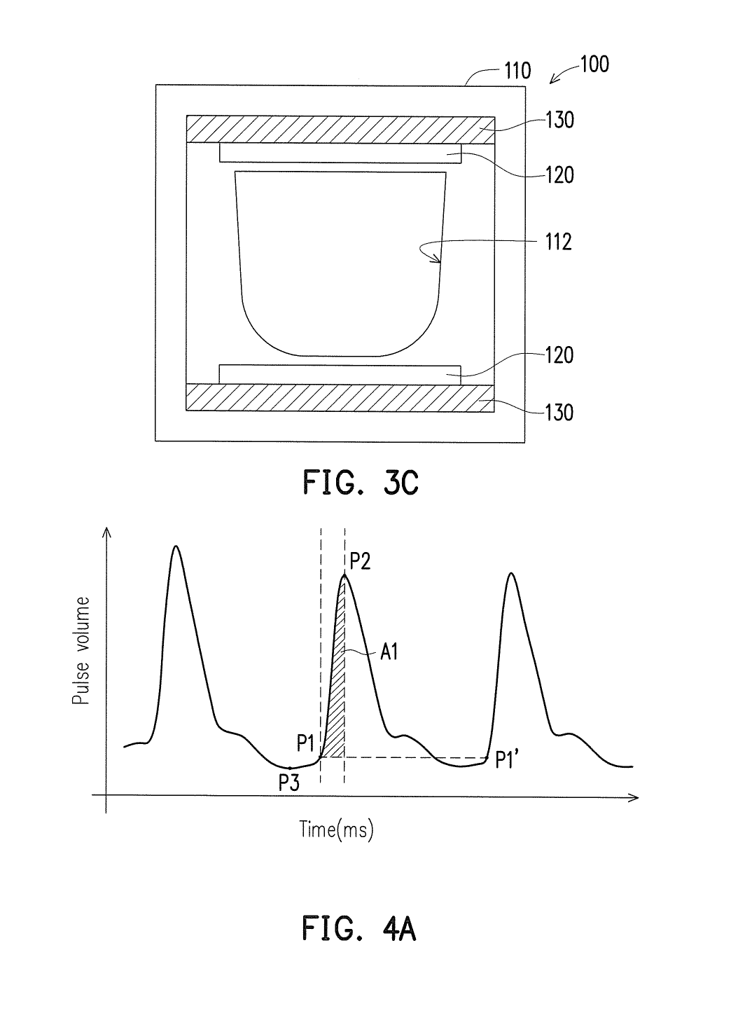

FIG. 2 is a block diagram of a physiological detection device according to an embodiment of the invention. FIG. 3A is a schematic view of the physiological detection device of FIG. 2. FIG. 3B and FIG. 3C are schematic side views of the physiological detection device of FIG. 3A from different aspects. Referring to FIG. 2 and FIG. 3A to FIG. 3C, in this embodiment, a physiological detection device 100 includes a main body 110, a sensor pair 120, a signal processor 130, and a calculation module 140. The sensor pair 120 is disposed in the main body 110 and adapted to detect a detected portion 50 of a human body. In this embodiment, the sensor pair 120 is a photoplethysmograph, for example, and detects and assesses a physiological state of the detected portion 50 of the human body by measuring light of a specific wavelength that the sensor pair 120 emits and receives, and an amount of absorbed spectral energy.

For instance, the detected portion 50 is a peripheral part of the human body, such as finger, toe, and earlobe. Referring to FIG. 2, in this embodiment, the sensor pair 120 includes a set or multiple sets of (one set is depicted in FIG. 2 as an example) an optical emitter 122 and an optical receiver 124. The optical emitter 122 and the optical receiver 124 may be light transmissive type or light reflective type. In this embodiment, a light emitted by the light transmissive optical emitter 122 reaches the optical receiver 124 after passing through the detected portion 50 of the human body. Additionally, in a case where the optical emitter 122 is the light reflective type, the light emitted by the optical emitter 122 is reflected to the optical receiver 124 by the detected portion 50 after reaching the detected portion 50.

In this embodiment, the optical emitter 122 and the optical receiver 124 are an infrared optical emitter and an infrared optical receiver capable of emitting and receiving an infrared light, for example. The emitted and received light has a wavelength in a range of 760 nm to 1 μm. However, this embodiment is not intended to limit the range of the wavelength of the light emitted and received by the sensor pair 120. In some other embodiments, the light of the optical emitter 122 and the optical receiver 124 may be a green light having a wavelength in a range of 495 nm to 570 nm. Alternatively, the light of the optical emitter 122 and the optical receiver 124 may be a red light having a wavelength in a range of 620 nm to 750 nm.

As shown in FIG. 2, the sensor pair 120 obtains a sensing signal S1 from the detected portion 50 of the human body. The sensing signal S1 is a PPG signal emitted by the photoplethysmograph, for example. For instance, a hemoglobin concentration of the blood of the human body may be deemed as a constant value, and the amount of hemoglobin in blood vessels is positively correlated with the blood volume. Therefore, the sensor pair 120 detects the amount of spectral energy absorbed by the hemoglobin of the blood in the detected portion 50 to infer a change of the blood volume in the blood vessels and thereby obtain the sensing signal S1.

As the above-mentioned, since the blood volume in the blood vessels of the human body increases and decreases periodically with systole and diastole, the amount of the spectral energy of the light absorbed by the blood also changes periodically with the heart beat. Thus, after the light is received by the optical receiver 124 of the sensor pair 120, the sensing signal S1 having a quasi-periodic change is generated.

Specifically, during systole, blood is pushed into the arteries from the ventricle. As the blood volume in the blood vessels increases, the amount of the spectral energy of the light absorbed by the blood increases and accordingly the sensing signal S1 of the sensor pair 120 changes. Therefore, the change of the sensing signal S1 of the sensor pair 120 and the blood volume (perfusion flow) in the blood vessels of the detected portion of the human body are correlated with each other.

Further, referring to FIG. 2 and FIG. 3A to FIG. 3C, in this embodiment, the signal processor 130 is disposed in the main body 110 and coupled to the sensor pair 120 to receive the sensing signal S1 generated by the sensor pair 120. The signal processor 130 includes a filter 132, an amplifier 134, and an analog-to-digital converter 136. In this embodiment, the filter 132 performs bandpass filtering on the sensing signal S1 received by the signal processor 130, and a filter frequency is in a range of 0.5 Hz to 5 Hz, for example. In this embodiment, the range of the filter frequency of the filter 132 may be adjusted as appropriate according to the requirements of measurement of the physiological detection device 100.

The amplifier of the signal processor 130 controls a gain of the sensing signal S1 to be appropriate automatically. Moreover, the analog-to-digital converter 136 converts the sensing signal S1, which is an analog signal, to a digital physiological signal S2 to facilitate the subsequent signal processing and related calculation.

In this embodiment, after signal gain control of the sensing signal S1 is performed by the amplifier 134, the sensing signal S1 is converted to the digital physiological signal S2 by the analog-to-digital converter 136. An order of processing the sensing signal S1 may be adjusted as appropriate according to the actual requirements. For example, the sensing signal S1 may be converted to the digital physiological signal S2 by the analog-to-digital converter 136 first, and then the signal is amplified by the amplifier 134.

The calculation module 140 is disposed in the main body 110 and coupled to the signal processor 130 to receive the digital physiological signal S2 processed by the signal processor 130. In this embodiment, the calculation module 140 performs calculation on the digital physiological signal S2 to obtain information of feature points of the digital physiological signal S2.

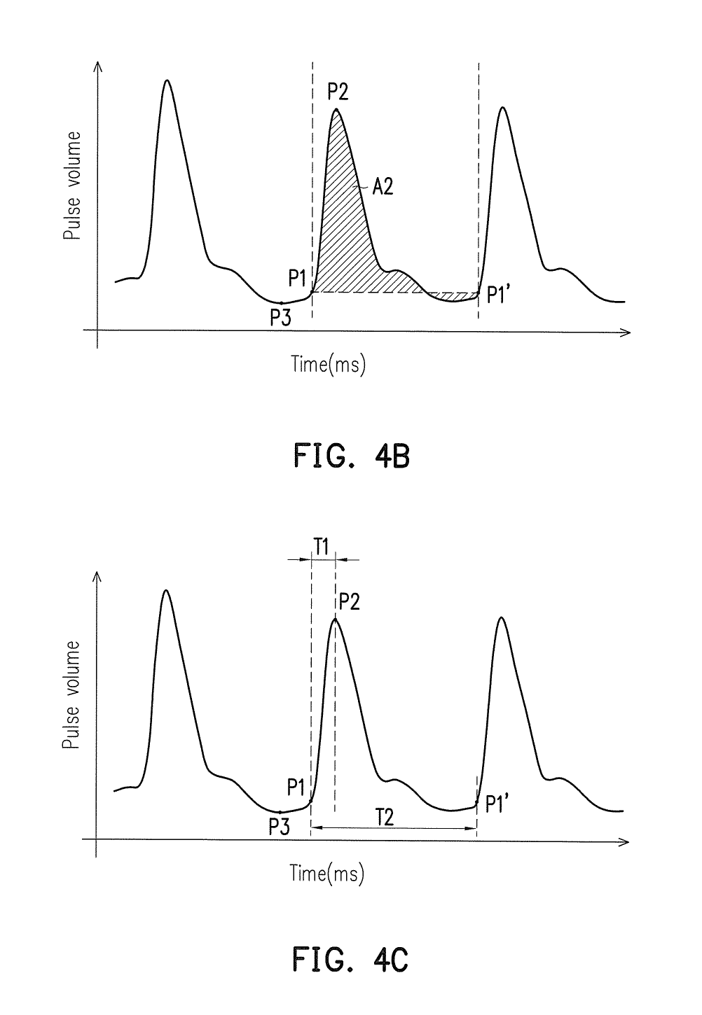

FIG. 4A to FIG. 4C are pulse waveform diagrams showing a pulse wave volume of the digital physiological signal of the physiological detection device of FIG. 2. Specifically, referring to FIG. 4A to FIG. 4C, in this embodiment, corresponding to the heart beat, the blood is periodically pushed into the blood vessels from the ventricle, and the digital physiological signal S2 has a plurality of pulse waves generated according to a time sequence.

In this embodiment, the pulse waves of the digital physiological signal S2 have a foot point P1 at a forepart of a rising edge, a pulse peak P2, and a trough point P3, which are feature points of the digital physiological signal S2.

In this embodiment, the foot point P1 of the digital physiological signal S2 reflects changes of pressure and volume in the blood vessels when diastole ends and systole is to begin. The pulse peak P2 is the highest point of the pulse waves and reflects a maximum pulse wave amplitude caused by the blood pushed into the blood vessels from the ventricle during systole. In this embodiment, the rise from the foot point P1 to the pulse peak P2 indicates a process of rapid expansion of the vascular wall as the intravascular blood volume in the artery increases rapidly when the blood is rapidly injected from the heart ventricle. The drop after the pulse peak P2 reflects a process that the blood volume in the arteries gradually decreases and the blood vessel walls are gradually restored to the state before expansion.

Referring to FIG. 2 again, in this embodiment, the calculation module 140 includes a normalization processor 142 and a physiological state index calculator 144. When the calculation module 140 completes calculation of the feature points of the digital physiological signal S2, the calculation module 140 normalizes the digital physiological signal S2 by the normalization processor 142 to restore the digital physiological signal S2 to the original signal before the signal gain. Then, the physiological state index calculator 144 calculates a physiological state index according to first information and second information of the feature points of the digital physiological signal S2.

Specifically, referring to FIG. 4A and FIG. 4B, the horizontal axis of the waveform of the pulse wave of the digital physiological signal S2 in FIG. 4A and FIG. 4B is the time axis, and a unit of the time is millisecond (ms). The vertical axis indicates the amplitude of the volume pulse wave of the digital physiological signal. In this embodiment, the first information of the digital physiological signal S2 is an integral area A1 of the pulse wave between the foot point P1 and the pulse peak P2 with respect to the time axis in FIG. 4A, and the second information is an integral area A2 of the pulse wave between adjacent two foot points P1 and P1′ with respect to the time axis in FIG. 4B. The physiological state index calculator 144 of the calculation module 140 calculates a ratio of the second information to the first information, i.e., a ratio of the integral area A2 to the integral area A1, to obtain the corresponding physiological state index.

Referring to FIG. 4C, in another embodiment, the first information is a time difference T1 between the foot point P1 and the pulse peak P2 in FIG. 4C, and the second information is a time difference T2 between the adjacent two foot points P1 and P1′ in FIG. 4C. The calculation module 140 may also calculate a ratio of the second information to the first information, i.e., a ratio of the time difference T2 to the time difference T1, to obtain the corresponding physiological state index.

In this embodiment, the user of the physiological detection device 100 may assess the state of blood perfusion in the blood vessels of the detected portion and the condition of blood circulation of the whole human body based on the physiological state index obtained through calculation of the calculation module 140.

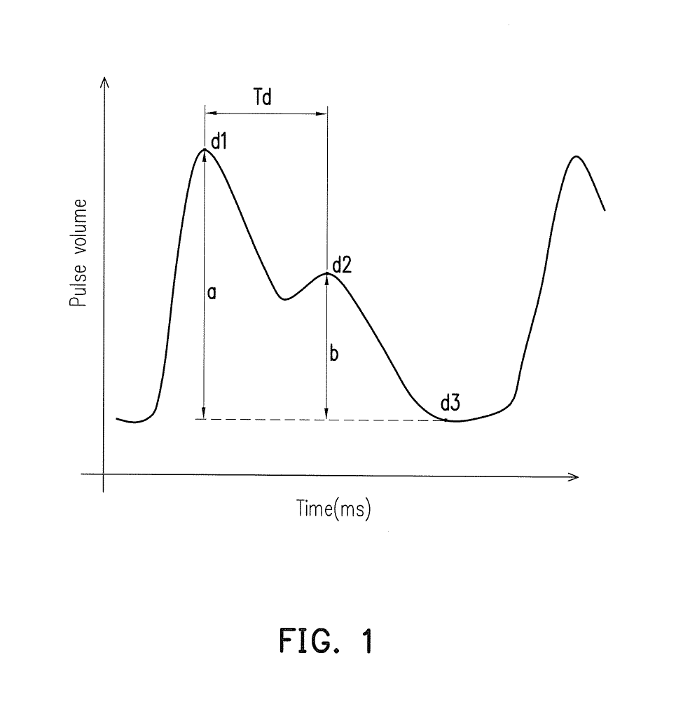

In comparison with the conventional technology shown in FIG. 1, when calculating the physiological state index, the physiological detection device 100 of this embodiment does not rely on the diastolic waves among the pulse waves of the digital physiological signal S2 of the subject to object the second information. Particularly, for subjects who are aged or in poor health, the pulse waves of the obtained digital physiological signal S2 may not include diastolic waves or the diastolic waves may not have obvious vertices. Consequently, the calculation module 140 of the physiological detection device 100 may not be able to effectively obtain the second information from the pulse waves of the digital physiological signal S2 to calculate the ratio of the second information and the first information and obtain the physiological state index.

In this embodiment, the second information is the pulse wave directly extracted between the adjacent two foot points P1 and P1′. That is, in this embodiment, the physiological detection device 100 obtains the second information directly from one complete cycle of pulse wave. Therefore, in addition to obtaining the second information from the pulse wave between the adjacent two foot points P1 and P1′, the physiological detection device of this embodiment may also obtain the second information from the pulse wave between any feature points (e.g., the trough point P3 in FIG. 4A) that appear repeatedly on adjacent pulse waves. Thus, the physiological detection device 100 of this embodiment obtains and calculates the second information in a simpler way than the conventional technology and is not limited to using the diastolic waves among the pulse waves and the vertices of the diastolic waves as the conventional technology of FIG. 1.

Furthermore, as compared with the conventional technology of FIG. 1, in addition to obtaining the first information and the second information from the time differences between the foot point P1 and the pulse peak P2 and between the two foot points P1 and P1′ to obtain the physiological state index, the physiological detection device 100 of this embodiment may also obtain the first information and the second information from the integral areas of the pulse waves between the foot point P1 and the pulse peak P2 and between the two foot points P1 and P1′ with respect to the time axis, so as to obtain the corresponding physiological state index through calculation.

The physiological detection device 100 of this embodiment may obtain the first information, the second information, and the physiological state index by the two methods described above, and compare the data obtained by the two methods to more accurately determine the condition of blood circulation of the human body.

Further, referring to FIG. 3A to FIG. 3C, in this embodiment, the main body 110 has a socket 112 for receiving the detected portion 50, e.g., a finger, of the user for detection. Moreover, a cushioning pad 114 is disposed on a socket wall of the socket 112 of the main body 110 to serve as a proper cushion between the finger and the main body 110 when the user inserts the finger into the main body 110. The cushioning pad 114 may be in the form of a clip or be replaceable, such that the user's finger is closely covered but not pressed when inserted into the socket 112.

In this embodiment, the physiological detection device 100 includes a display 150 disposed on a surface of the main body 110 to display the physiological state index that the calculation module 140 obtains through calculation. The display 150 is, for example, a seven-segment display. Nevertheless, this embodiment is not limited thereto. The physiological detection device 100 may also use an organic light emitting diode (OLED) or other display elements as the display 150.

Then, referring to FIG. 1 and FIG. 3A, the main body 110 of the physiological detection device 100 includes a printed circuit board (PCB) 117, on which an alarm 160 is disposed. The alarm 160 includes a light emitting diode (LED) 162 and a buzzer 164. The light emitting diode 162 and the buzzer 164 respectively generate a light or a sound as an alarm when the physiological state index of the subject exceeds a set standard value. Alternatively, when the system of the physiological detection device 100 has a malfunction, the physiological detection device 100 may send a signal indicating the system malfunction through the light emitting diode 162 or the buzzer 164. In addition, the printed circuit board 117 may be replaced by a flexible printed circuit (FPC).

The physiological detection device 100 includes a power supply 170, which includes a switch button 172 and a power supply module 174. In this embodiment, supply of power to the physiological detection device 100 is turned on or off by pressing the switch button 172. Moreover, the power supply module 174 of the physiological detection device 100 is electrically connected with the sensor pair 120, the signal processor 130, and the calculation module 140 to provide power for operation. Furthermore, the power supply module 174 may be a rechargeable battery or a disposable alkaline battery. This embodiment is not intended to limit the type of power supply of the power supply module 174.

In this embodiment, the physiological detection device 100 further includes a transmitter 180, e.g., Bluetooth, WiFi, or USB, disposed on the printed circuit board 117 for transmitting the physiological state index to an external device, such as a smart phone, a tablet computer, or a remote server, that is capable of displaying and recording data. Moreover, the physiological detection device 100 may be connected with other physiological detection devices 100 or electrically connected with an external power supply through the transmitter 180.

The physiological detection device 100 further includes a memory 190 disposed on the printed circuit board 117. The memory 190 is a data storage device, e.g., a flash memory, for storing the obtained sensing signal S1 and physiological state index.

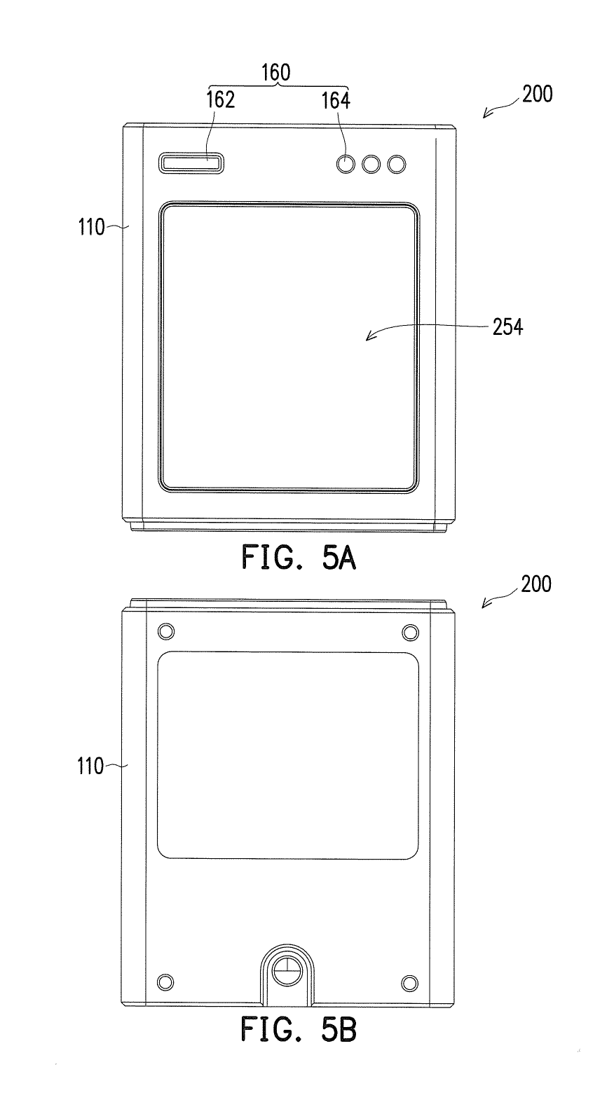

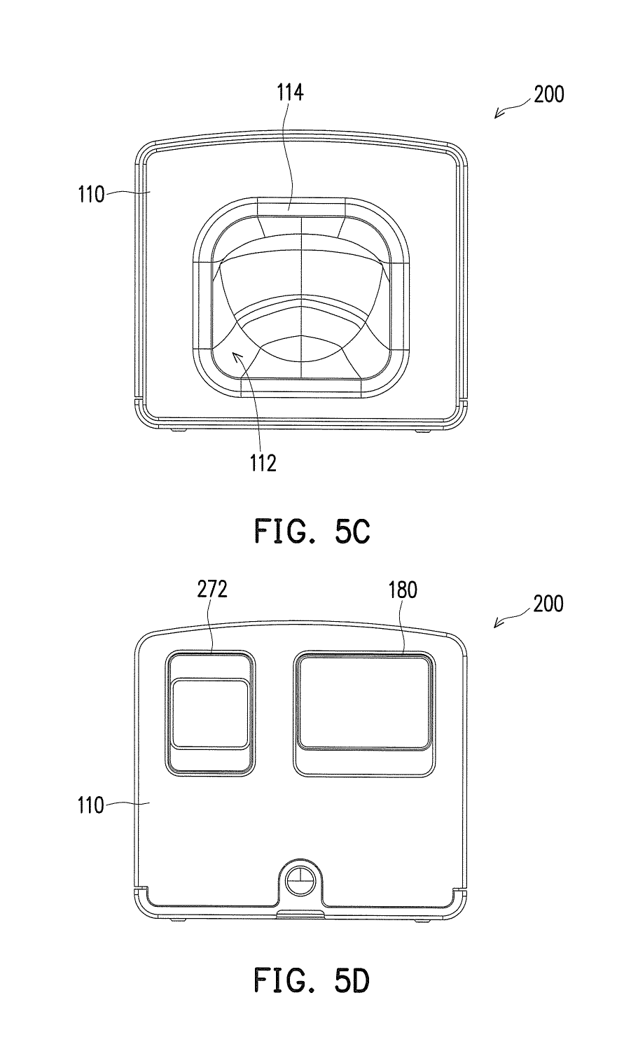

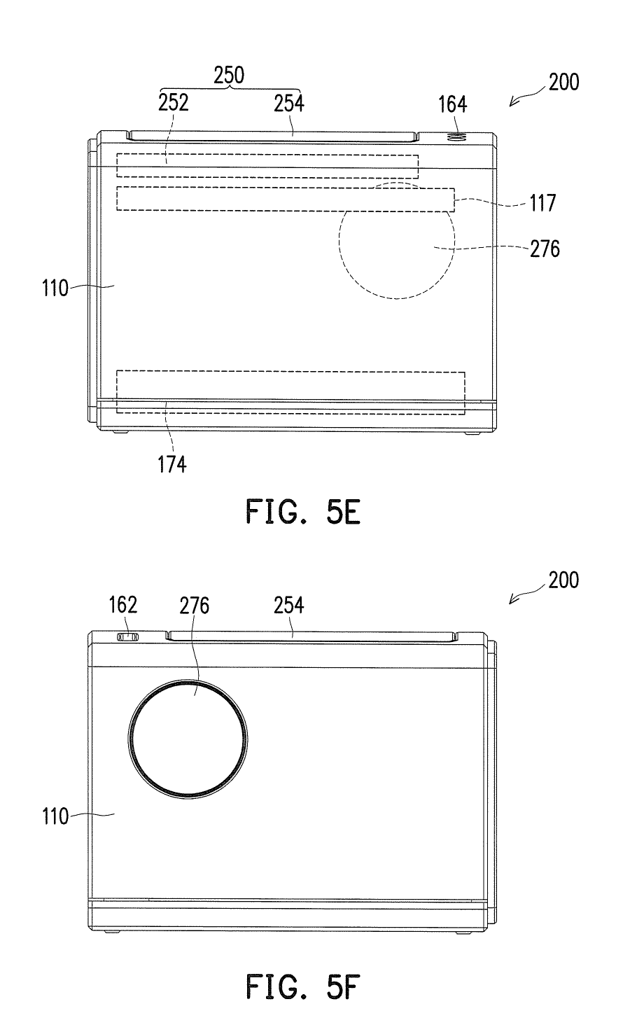

FIG. 5A to FIG. 5F are schematic views of appearance of a physiological detection device 200 according to another embodiment of the invention, wherein FIG. 5A and FIG. 5B are top and bottom views of the physiological detection device 200, and FIG. 5C, FIG. 5D, FIG. 5E, and FIG. 5F are side views of the physiological detection device 200 from different aspects. The physiological detection device 200 of this embodiment is similar to the physiological detection device 100 in structure. Therefore, identical or similar components are denoted by using the same or similar reference numerals and details thereof are not repeated hereinafter. Referring to FIG. 5A to FIG. 5F, in this embodiment, a display 250 of the physiological detection device 200 is disposed on an upper surface of the main body 110. The display 250 includes a display device 252 and a covering glass 254. The covering glass 254 protects the display device 252, and the user may see a message displayed by the display device 252 through the covering glass 254.

Referring to FIG. 3A, FIG. 5D, and FIG. 5F, in contrast to the physiological detection device 100 in which the switch button 172 is disposed on the upper surface of the main body 110, the number and configuration of the switch buttons 272 and 276 of this embodiment may be adjusted and changed according to the actual application and functional requirements. For example, as shown in FIG. 5D and FIG. 5F, the switch buttons 272 and 276 of the physiological detection device 200 are disposed on different side surfaces. In addition, the switch button 272 in FIG. 5D is used for controlling the power supply of the entire physiological detection device 200 while the switch button 276 in FIG. 5F is used for turning on and off the display 250, for example. This embodiment is not intended to limit the number, configuration, and functions of the switch buttons 272 and 276.

To sum up, the physiological detection device of the embodiments of the invention utilizes the optical emitter of the physiological detection device to emit light, which passes through the detected portion of the human body or is reflected to the optical receiver of the physiological detection device by the detected portion, so as to obtain the sensing signal. Moreover, the sensing signal is processed by the signal processor to obtain the digital physiological signal. The physiological detection device of the invention calculates the ratio of the integral area of the pulse wave of one full cycle with respect to the time axis to the integral area of the pulse wave from the foot point to the pulse peak with respect to the time axis based on the foot point and the pulse peak of the pulse wave of the digital physiological signal, so as to obtain the corresponding physiological state index. Moreover, the physiological detection device of the invention calculates the ratio of the time difference between two foot points (i.e., time of the full pulse wave cycle) to the time difference from the foot point to the pulse peak of the pulse wave, so as to obtain the corresponding physiological state index. Accordingly, the way the physiological detection device of the invention obtains the physiological state index is not limited by whether the pulse waves of the subject include diastolic waves and locations of the vertices of the diastolic waves, for the subject to easily and quickly obtain the physiological state index and thereby assess the condition of blood circulation.

It will be apparent to those skilled in the art that various modifications and variations can be made to the disclosed embodiments without departing from the scope or spirit of this invention. In view of the foregoing, it is intended that the invention covers modifications and variations provided that they fall within the scope of the following claims and their equivalents.

What is claimed is:

1. A physiological detection device, comprising:
a main body;
a sensor pair disposed in the main body and detecting a detected portion of a human body to obtain a sensing signal;
a signal processor disposed in the main body and receiving and processing the sensing signal to output a digital physiological signal; and
a calculation module receiving the digital physiological signal and calculating to obtain first information and second information of a plurality of feature points of the digital physiological signal, and calculating a ratio of the second information to the first information to obtain a physiological state index, wherein the digital physiological signal comprises a plurality of pulse waves generated according to a time sequence, and the feature points of the digital physiological signal comprise a pulse peak of each of the pulse waves and a foot point at a forepart of a rising edge of each of the pulse waves.

2. The physiological detection device according to claim 1, wherein the first information is an integral area of the pulse wave between the foot point and the pulse peak with respect to a time axis while the second information is an integral area of the pulse wave between adjacent two foot points with respect to the time axis.

3. The physiological detection device according to claim 1, wherein the first information is a time difference between the foot point and the pulse peak while the second information is a time difference between the adjacent two foot points.

4. The physiological detection device according to claim 1, wherein the sensor pair is a photoplethysmograph, comprising:
an optical emitter emitting a light that passes through the detected portion of the human body; and
an optical receiver receiving the light passing through the detected portion to obtain the sensing signal.

5. The physiological detection device according to claim 1, wherein the signal processor comprises:
a filter performing filtering on the sensing signal;
an amplifier amplifying the sensing signal; and
an analog-to-digital converter converting the sensing signal into the digital physiological signal.

6. The physiological detection device according to claim 1, wherein the calculation module comprises:
a normalization processor normalizing the digital physiological signal; and
a physiological state index calculator calculating the physiological state index according to the first information and the second information of the feature points of the normalized digital physiological signal.

7. The physiological detection device according to claim 1, further comprising an alarm disposed in the main body and electrically connected with the calculation module.

8. The physiological detection device according to claim 1, further comprising a display disposed on a surface of the main body and displaying the physiological state index.

9. The physiological detection device according to claim 1, further comprising a power supply disposed in the main body and electrically connected with the sensor pair, the signal processor, and the calculation module.

10. The physiological detection device according to claim 1, further comprising a transmitter disposed in the main body and transmitting the physiological state index outside the physiological detection device.
